(19) United States
(12) Patent Application Publication
(10) Pub. No.: US 2010/0010259 A1
(43) Pub. Date: Jan. 14, 2010

(54) Process for producing anhydrous peroxydisulfuric, acid using sulfur trioxide recovered from a waste comprising sulfuric acid

(76) Inventor: Roy William Martin, Downers Grove, IL (US)

Correspondence Address: Roy W. Martin, 1440 Palmer, Downers Grove, IL, 60516, US

(21) Appl. No.: 12/217,681

(22) Filed: Jul. 8, 2008

(51) Int. Cl. C07C 309/03

(52) U.S. Cl. 562/122

(57) ABSTRACT

A process for producing peroxydisulfuric acid from a waste comprising substantially sulfuric acid, whereby the sulfuric acid is separated into sulfur trioxide and substantially water which is removed as a waste. The recovered sulfur trioxide is reacted with hydrogen peroxide to produce peroxydisulfuric acid which is fed to a methane-sulfonation process resulting in formation of sulfuric acid radicals. The sulfuric acid radicals react with methane producing methane radicals resulting in formation of sulfuric acid. The sulfuric acid from the methane-sulfonation process is recovered, reprocessed, and the process is repeated.

### FIELD OF INVENTION

This Invention relates to the process for producing an anhydrous peroxydisulfuric acid for use in a methane-sulfonation process, whereby the peroxydisulfuric acid is produced from reacting hydrogen peroxide or Caro's acid with sulfur trioxide removed and recovered from a methane-sulfonation process waste stream comprising substantially sulfuric acid. The invention provides a means for continuous production of peroxydisulfuric acid from recycled sulfur trioxide recovered from a waste comprising substantially sulfuric acid while producing a waste comprised of substantially water.

### BACKGROUND

The methane-sulfonation processes disclosed in U.S. Pat. No. 7,282,603 (“the '603 Patent”) requires an initiator such as peroxydisulfuric acid H₂S₂O₈ to generate a sulfate free radical having the general formula HSO₄*, where the asterisk represents a missing or unpaired electron.

During the in-situ generation of peroxydisulfuric acid using sulfur trioxide (SO₃) and aqueous hydrogen peroxide (H₂O₂), sulfuric acid is produced from the reaction of SO₃ and H₂O that accompanies the hydrogen peroxide:

3SO₃+H₂O₂+H₂O>>>>H₂S₂O₈+H₂SO₄

Furthermore, the process of converting methane to methane radicals by reaction with a sulfuric acid radical or sulfate free radical disclosed in U.S. Pat. No. 7,282,603 results in removal of a hydrogen from the methane and subsequent formation of sulfuric acid. The resulting sulfuric acid (H₂SO₄) is a byproduct that will increase in concentration in the cyclic process unless it is removed from the process loop.

U.S. Pat. No. 3,927,189 discloses a process for producing peroxydisulfuric acid and its salts by reacting sulfur trioxide with either Caro's acid or hydrogen peroxide under controlled conditions.

U.S. Pat. No. 3,939,072 (“the '072 Patent”) teaches a process for point of use production of Caro's acid, in which the Caro's acid is cooled to between −10° C. to 80° C. to reduce decomposition of the Caro's acid before its use.

U.S. Pat. No. 5,141,731 (“the '731 Patent”) teaches a process and an apparatus for point of use generation of peroxyacids by adding H₂O₂ to a stream of H₂SO₄ in multiple stages.

U.S. Pat. No. 5,429,812 (“the '812 Patent”), which discloses a process of producing peroxysulfuric acid from substoichiometric levels of H₂SO₄ to H₂O₂, teaches using a substoichiometric amount of H₂SO₄ to produce an equilibrium amount of Caro's acid. The final mixture in the '812 Patent has a molar ratio of SO₃ to Available Oxygen in the range of 0.8 to 0.2.

U.S. Pat. No. 5,139,763 (“the '763 Patent”) teaches making Caro's acid with a supra-stoichiometric molar amounts of oleum to H₂O₂.

U.S. patent application Ser. No. 10/878,176 now abandoned disclosed a thin-film reactor for producing Caros' acid.

U.S. Pat. No. 7,390,418 B2 (“the '418 Patent”) discloses a process for producing Caro's acid with substantially no peroxydisulfuric acid and high yield of Caro's acid.

There are many processes for producing Caro's acid from hydrogen peroxide and sulfuric acid, oleum, and sulfur trioxide. Prior art also discloses methods for reacting either hydrogen peroxide or Caro's acid with sulfur trioxide to produce peroxydisulfuric acid.

As either stand alone processes or used in any combination, the disclosed processes can not be retrofitted to the methane-sulfonation process disclosed in U.S. Pat. No. 7,282,603 without resulting in the generation of sulfuric acid that will concentrate in a cyclic process. As a result the sulfuric acid waste would require removal from the process and be handled as waste. The application of any combination of the disclosed processes to produce peroxydisulfuric acid would increase the methane-sulfonation processing cost by having to replenish spent reactants such as SO₃ lost as sulfuric acid, as well as the cost of neutralization or the storage, handling, and waste removal of the sulfuric acid.

### SUMMARY

Therefore a unique process has been developed wherein a product rich in anhydrous peroxydisulfuric acid is produced using a processed waste stream recovered from the methane-sulfonation process that comprises substantially sulfuric acid.

The process comprises: a) heating to a mother liquor comprising substantially sulfuric acid to volatilize at least the sulfur trioxide, b) separating the volatilized sulfur trioxide from substantially water using distillation, c) removing substantially water from the process while recovering the sulfur trioxide , d) feeding hydrogen peroxide into a jacketed reactor while feeding at least the recovered sulfur trioxide into said jacketed reactor to obtain at least stoichiometric levels of sulfur trioxide based on the total flow-rate of hydrogen peroxide and water, e) reacting the sulfur trioxide and hydrogen peroxide while sustaining temperature that stabilizes the resulting anhydrous peroxydisulfuric acid, f) feeding the solution comprising peroxydisulfuric acid into the methane sulfonation process, g) recovering the stream from the methane sulfonation process comprising substantially sulfuric acid, h) and, repeating the process.

### BRIEF DESCRIPTION OF THE DRAWINGS

FIG. 1 is a schematic illustration of a cyclic process for producing peroxydisulfate from a recovered waste stream comprising sulfuric acid from the methane-sulfonation process.

FIG. 2 is a schematic illustration of a Stirred Tank jacketed reactor for producing peroxydisulfiric acid

FIG. 3 is a schematic illustration of a Centrifugal or Rotating jacketed reactor for producing peroxydisulfuric acid.

FIG. 4 is a schematic illustration of a distillation process for recovering sulfur trioxide from sulfuric acid.

FIG. 5 is a graph illustrating the mole fractions of constituents resulting from the thermal decomposition of sulfuric acid.

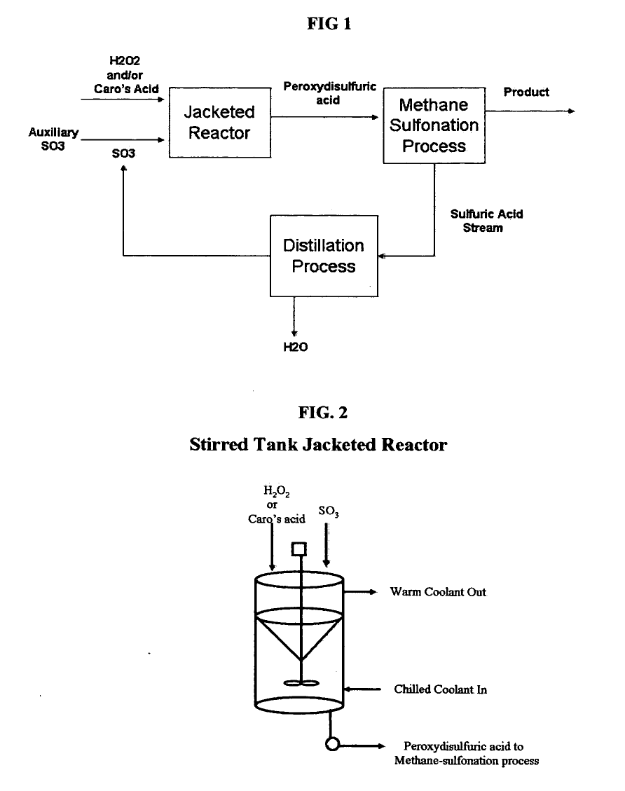

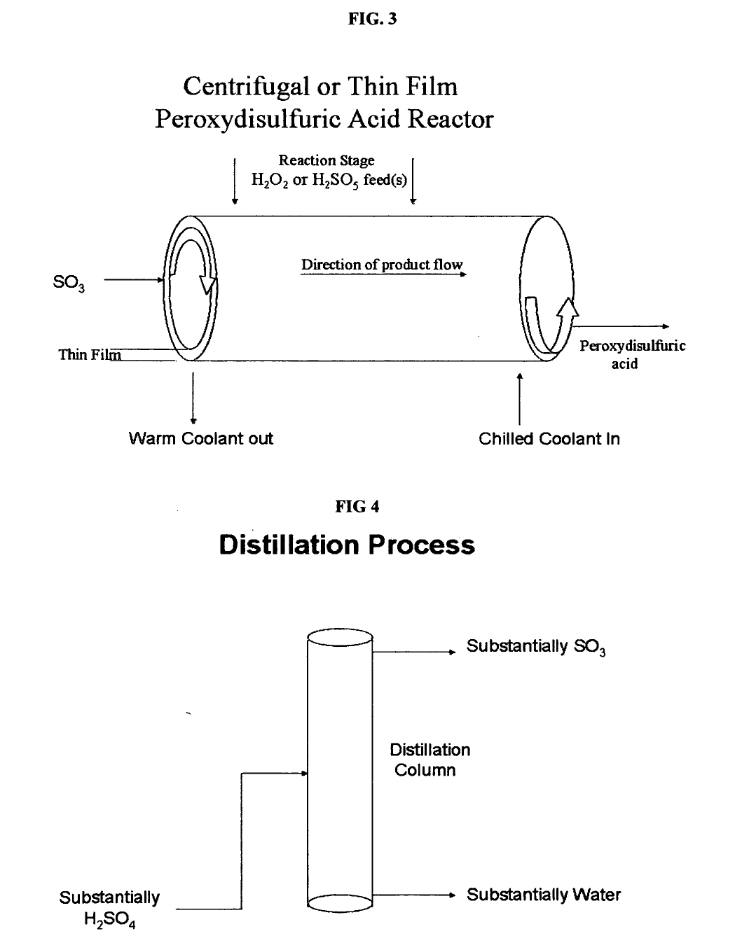

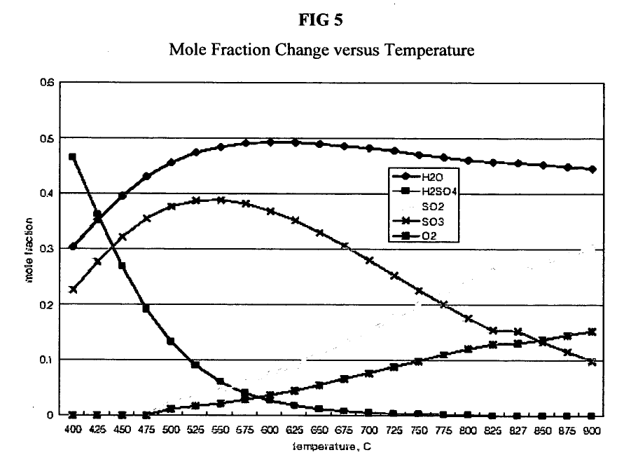

### DETAILED DESCRIPTION OF THE PREFERRED EMBODIMENT(S)

Embodiments of the invention are described herein in the context of a process for producing anhydrous peroxydisulfuric acid from a supply of sulfuric acid recovered from the methane-sulfonation process disclosed in U.S. Pat. No. 7,282,603, and particularly in the context of a cyclic process for producing the peroxydisulfuric acid.

Yet another embodiment is a cyclic process for producing a sulfuric acid radical initiator comprising peroxydisulfuric acid from a recovered byproduct comprising sulfuric acid resulting from reaction of sulfuric acid radical(s) with hydrogen(s) removed from methane(s) and resulting in a methane radical(s).

It is to be understood that the embodiments provided herein are just preferred embodiments, and the scope of the invention is not limited to the applications or the embodiments disclosed herein. For example, although the sulfuric acid source for recovery of sulfur trioxide and subsequent production of peroxydisulfuric acid is from a methane-sulfonation process, the same cyclic process for producing peroxydisulfuric acid can be applied using any recovered source of sulfuric acid such as other types of organic-sulfonation processes employing peroxydisulfuric acid as an initiator for producing sulfate free radicals.

As used herein:

“Hydrogen peroxide” includes anhydrous H₂O₂ comprising 100 wt % H₂O₂, aqueous H₂Ocomprising 30 wt % to 99.9 wt % H₂O₂, and Caro's acid comprising residual H₂O₂.

“Mother liquor” is any liquid comprising at least some portion of sulfuric acid that is fed to the distillation system for separation of the SO₃ from the sulfuric acid. The mother liquor can be a mix of process waste streams resulting from any of the process steps in the methane-sulfonation process as well as any recycled stream from the distillation process that comprises sulfuric acid.

“Methane-sulfonation process” is any sequential process steps required for the sulfonation of methane to produce at least methane-sulfonic acid (MSA) or when applicable its surrogate end products, the said process including but not limited to: generating sulfuric acid radicals from peroxydisulfuric acid, the preparation and feed of methane and sulfur trioxide to the reactor used to produce MSA, contacting the sulfuric acid radicals with methane to produce methane radicals, reacting the methane radicals with sulfur trioxide to produce MSA, recovery of MSA and a waste stream comprising substantially sulfuric acid and/or continued processing of MSA releasing sulfur dioxide and methanol, recovery and processing of sulfur dioxide resulting in formation of sulfur trioxide, and recovery and storage of the sulfur trioxide.

“Distillation” is any technique that evaporates at least some portion of the mother liquor due to differences in their boiling points, separates the volatilized component(s) from the mother liquor, and condenses the vapor.

In “fractional distillation” the components comprising the mother liquor are separated due to differences in boiling point by repeating a cycle of evaporation-condensation at difference temperatures regions within a column. The column can be packed or include trays that induce lower boiling point components to condense while contacting the condensate with hotter rising vapor(s). This cycling or reflux enhances separation of the components entering the column.

“Vacuum distillation” shall include any distillation method for separating at least sulfur trioxide from substantially sulfuric acid where a vacuum is sustained to separate a vapor from a liquid fraction. Vacuum distillation allows vapor-liquid separation at lower temperature and can be applied to fractional distillation.

“Substantially water” means the waste stream from the distillation process has had a major portion of the H₂SO₄ removed prior to being separated from the process, but does not necessarily mean there is no remaining H₂SO₄.

“Substantially sulfuric acid” is interchangeable with “sulfuric acid” and shall include oleum which comprising sulfuric acid with sulfur trioxide, as well as any other sulfur oxides and residual compounds from the methane-sulfonation process that are included with the recovered sulfuric acid.

“Anhydrous peroxydisulfuric acid” and “peroxydisulfuric acid” are interchangeable.

“Sulfate free radical” and “sulfuric acid radical” are interchangeable.

“Sustaining temperature that stabilizes the resulting peroxydisulfuric acid” refers to the range in temperature that provides sufficient stability as to not exceed an acceptable loss of peroxydisulfuric acid activity due to thermal decomposition resulting from prolonged exposure to elevated temperatures prior to application to the methane-sulfonation process. The allowable temperature range can vary based on the residence time in the jacketed reactor, as well as any residence time prior to being fed to the methane-sulfonation process. For example, a process that produces peroxydisulfuric acid at about 90° C. and applies the product directly to the methane sulfonation process with a total residence time of about 5 seconds will experience a very acceptable loss of product resulting from thermal decomposition. The same process requiring 240 seconds of residence time at about 90° C. will experience a substantial lose that will significantly impact cost effectiveness. However for comparison, the same process design that requires 240 seconds sustained at about 20° C. would provide very acceptable cost effectiveness due to improved stability and reduced thermal decomposition of the peroxydisulfuric acid.

Therefore, the sustaining temperature that stabilizes the resulting peroxydisulfuric acid should not allow more than 50 wt % decomposition of the total wt % peroxydisulfuric acid produced in the jacketed reactor. Preferably the sustaining temperature that stabilizes the resulting peroxydisulfuric acid should not allow more than 20 wt % decomposition of peroxysulfuric acid from the maximum wt % produced in the jacketed reactor, and most preferred the sustaining temperature that stabilizes the resulting peroxydisulfuric acid results in less than 10 wt % decomposition of peroxydisulfuric acid from the maximum wt % produced in the jacketed reactor.

“Cyclic process” refers to recovering and reusing at least a portion of the sulfur trioxide used to produce peroxydisulfuric acid from the sulfuric acid recovered from the methane-sulfonation process.

“Jacketed Reactor” is interchangeable with “centrifugal reactor”, “stirred tank reactor”, and in the context of this application will be defined as any reactor design or configuration that has a means of reacting at least the sulfur trioxide and hydrogen peroxide as disclosed while removing heat from the exothermic reaction at a sufficient rate as to be able to control the temperature of the reacting solution and resulting product. The jacketed reactor allows cooling media that is segregated from the reactants and resulting peroxydisulfuric acid to remove heat thereby controlling temperature and improving product stability. Jacketed reactors are common throughout the petrochemical, pharmaceutical, and food processing industries and to those skilled in the art, and can be configured into numerous reactor designs. A Jacketed Reactor may be a coil inside a reactor vessel, a coil between two shells, compartments along the axes of the reactor where coolant enters, is heated, and leaves as warm coolant.

“Total residence time” includes the time the hydrogen peroxide and sulfur trioxide are introduced into the jacketed reactor and includes the time the peroxydisulfuric acid is introduced to the methane-sulfonation process.

The process for producing peroxydisulfuric acid from a recovered waste stream comprising substantially sulfuric acid has two primary steps. The first step is producing the peroxydisulfuric acid, followed by processing the recovered waste stream comprising substantially sulfuric acid to separate the sulfur trioxide for reuse, and produce a waste comprising substantially water that is removed from the process.

Production of peroxydisulfuric acid can utilize various types of reactors. Without intent of limiting the variations in reactor type and design, some examples include a Stirred Tank Reactor and Centrifugal Reactor. Examples of these types of reactors and their application for producing various peroxyacids are included in the referenced prior art. Which reactor is best suited to the production of peroxysulfuric acid applied to the methane-sulfonation process can vary based on numerous factors. For example, a centrifugal reactor provides excellent heat transfer, low residence time, and compact design, while a stirred tank reactor is low cost but requires increased residence time.

The temperature within the jacketed reactor should not be higher than about 90° C., preferably no higher than 45° C., and most preferably no higher than 20° C. For further clarification and without limiting the scope of the process, the actual allowable temperature range to sustain product stability is dependent on the type of jacketed reactor, the total residence time beginning from the time hydrogen peroxide and sulfur trioxide are introduced to the jacketed reactor to the time the peroxydisulfuric acid is introduced to the methane-sulfonation process. For example, a centrifugal reactor with a residence time measured in seconds followed by immediate application to the methane-sulfonation process allows for higher temperature processing thereby reducing cooling cost and reducing or altogether eliminating heating the peroxydisulfuric acid as part of the conversion to sulfuric acid radicals disclosed in ‘603’. However, a stirred tank reactor having higher residence times as compared to a centrifugal reactor will require lower processing temperatures to ensure stability of the resulting peroxydisulfuric acid.

Distillation can be a simple as a heating the sulfuric acid to its fuming temperature, flashing the volatiles by pressure drop in a flash tank, followed by condensing of the volatiles comprising substantially sulfur trioxide. This process can be performed in series using varying temperatures and pressure drops based on the changing composition of the feed to the flash tank.

A more practical method is to employ a fractionation column that has been designed for processing a feed stream comprising substantially sulfuric acid. One such system is described by Dr. Youngjoon Shin in “A Dynamic Study on the Sulfuric Acid Distillation Column for VHTR-assisted Hydrogen Production Systems” presented at the 2007 International Congress on Advances in Nuclear Power Plants, Nice Acropolis, France. In this report, Dr. Shin discloses a distillation column equipped with 2 ideal plates and having the 2^{nd} plate feeding system from the bottom plate.

Because the peroxydisulfuric acid feed rate relative to the feed-rate of methane is comparatively small, a relatively compact processing system can be designed to support a much larger methane-sulfonation process. Using the process steps disclosed, commercially available reagents such as aqueous hydrogen peroxide and sulfur trioxide can be utilized to produce peroxydisulfuric acid while recovering a waste stream comprising substantially sulfuric acid, recovering the sulfur trioxide from the sulfuric acid for reuse in the production of peroxydisulfuric acid, while removing a waste stream comprising substantially water.

I claim:

1) A process for producing peroxydisulfuric acid from a recovered methane-sulfonation process waste containing substantially sulfuric acid, the process comprising: a) heating a mother liquor comprising substantially sulfuric acid to volatilize at least the sulfur trioxide; b) separating the volatilized sulfur trioxide from the fraction comprising substantially water using distillation; c) removing the fraction of substantially water from the process while recovering the sulfur trioxide; d) feeding hydrogen peroxide into a jacketed reactor while feeding at least the recovered sulfur trioxide into said jacketed reactor to obtain at least stoichiometric levels of sulfur trioxide based on the total flow-rate of hydrogen peroxide and water; e) reacting the sulfur trioxide and hydrogen peroxide while sustaining temperature that stabilizes the resulting anhydrous peroxydisulfuric acid; f) and feeding the solution comprising peroxydisulfuric acid to the methane-sulfonation process.

2) A process of claim 1 wherein the jacketed reactor is a stirred tank reactor.

3) A process of claim 1 wherein the jacketed reactor is a centrifugal reactor.

4) A process of claim 1 wherein distillation is achieved by means of fractional distillation.

5) A process of claim 1 wherein distillation is achieved by means of vacuum distillation.

6) A cyclic process for producing peroxydisulfuric acid from a recovered methane-sulfonation process waste comprising substantially sulfuric acid, the process comprising:
a) heating the mother liquor comprising substantially sulfuric acid to volatilize at least the sulfur trioxide; b) separating the volatilized sulfur trioxide from the fraction comprising substantially water using distillation; c) removing the fraction of substantially water from the process while recovering the sulfur trioxide; d) feeding hydrogen peroxide into a jacketed reactor while feeding at least the recovered sulfur trioxide into said jacketed reactor to obtain at least stoichiometric levels of sulfur trioxide based on the total flow-rate of hydrogen peroxide and water; e) reacting the sulfur trioxide and hydrogen peroxide while sustaining temperature that stabilizes the resulting anhydrous peroxydisulfuric acid; f) feeding the solution comprising peroxydisulfuric acid to the methane-sulfonation process; g) recovering the waste from the methane-sulfonation process comprising at least the sulfuric acid; h) and repeating the process.

7) A process of claim 6 wherein the jacketed reactor is a stirred tank reactor.

8) A process of claim 6 wherein the jacketed reactor is a centrifugal reactor.

9) A process of claim 6 wherein distillation is achieved by means of fractional distillation.

10) A process of claim 6 wherein distillation is achieved by means of vacuum distillation.

11) A cyclic process for producing a sulfuric acid radical initiator comprising peroxydisulfuric acid from a byproduct comprising sulfuric acid resulting from the reaction of sulfuric acid radicals with hydrogen comprising:
a) heating the mother liquor comprising substantially sulfuric acid to volatilize at least the sulfur trioxide; b) separating the volatilized sulfur trioxide from the fraction comprising substantially water using distillation; c) removing the fraction of substantially water from the process while recovering the sulfur trioxide; d) feeding hydrogen peroxide into a jacketed reactor while feeding at least the recovered sulfur trioxide into said jacketed reactor to obtain at least stoichiometric levels of sulfur trioxide based on the total flow-rate of hydrogen peroxide and water; e) reacting the sulfur trioxide and hydrogen peroxide while sustaining temperature that stabilizes the resulting anhydrous peroxydisulfuric acid; f) feeding the solution comprising peroxydisulfuric acid to the methane-sulfonation process; g) recovering the waste from the methane-sulfonation process comprising at least the sulfuric acid; h) and repeating the process.

12) A process of claim 11 wherein the jacketed reactor is a stirred tank reactor.

13) A process of claim 11 wherein the jacketed reactor is a centrifugal reactor.

14) A process of claim 11 wherein distillation is achieved by means of fractional distillation.

15) A process of claim 11 wherein distillation is achieved by means of vacuum distillation.
